(19) United States
(12) Patent Application Publication
(10) Pub. No.: US 2024/0096467 A1
(43) Pub. Date: Mar. 21, 2024

(54) SYSTEM AND METHOD FOR COLLECTING USER HEALTH DATA AND GENERATING, PRESENTING, AND REFINING HEALTH ANALYSIS AND RECOMMENDATIONS USING AN ELECTRONIC DEVICE

(71) Applicant: Finding Sally, Inc., Austin, TX (US)

(72) Inventors: Corey Suzanne Jackson, Austin, TX (US); Ralph Carlton Jackson, III, Austin, TX (US)

(73) Assignee: Finding Sally, Inc., Austin, TX (US)

(21) Appl. No.: 17/950,070

(22) Filed: Sep. 21, 2022

(51) Int. Cl. G16H 20/00; G16H 10/20

(52) U.S. Cl. CPC G16H 20/00; G16H 10/20

(57) ABSTRACT

An apparatus and method including receiving first user input information input by the first user that includes a beginning date and an end date of a most recent menstrual cycle of the first user. An upcoming menstrual cycle or a phase of an existing menstrual cycle is determined from first user input information. First bodily function information is received from a wearable device, and at least one of a recommended food or exercise regimen is generated based on the first bodily function information, the first user input information, and the determined upcoming menstrual cycle or determined phase of the existing menstrual cycle. Second user input information and second bodily function information is subsequently received, and a revised food or exercise regimen is generated based on the second bodily function information, the second user input information, and the upcoming menstrual cycle or phase of the existing menstrual cycle.

### BACKGROUND

### Field

This disclosure relates to applying health information and to a device and method for identifying menstrual cycles in perimenopausal women and applying health recommendations based on user health data, machine learning, and feedback.

### Description of the Related Art

People who menstruate go through a set of regular hormone cycles. There are a variety of factors—including genetic, socioeconomic, and environmental factors—that can change the cycle and the female's experience with it. Some of these factors can also predispose individuals to different conditions that impact the cycle. Most scientific literature on the topic of human menstruation describes study participants using terms such as woman, women, her, hers, etc. Those same terms are used here for consistency and readability. People who menstruate have the characteristics and needs described herein.

These menstrual cycles become less predictable later in life, leading up to the last period. Menopause is defined as the point in time at which twelve months have passed without a menstrual period, after which a woman is considered post-menopausal. There is also typically a transition period before menopause when a woman's menstrual cycles become less predictable. This transition period corresponds to the time between the onset of less predictable cycles and twelve months after the last cycle and is called perimenopause. For most women, perimenopause is a 10-to-15-year span but can be longer or shorter.

Perimenopausal and post-menopausal women can experience a host of around 34 known unpleasant symptoms of estrogen decline. These symptoms can impact a user's daily quality of life in ways that interfere with typical healthy lifestyle patterns. Symptoms include, for example, insomnia and disrupted sleep, which can impede recovery from previous exercise. A user's instinct might be to continue intense workouts despite the fatigue that results from impaired sleep quality, but that could put her at risk of acute or overuse injury due to inadequate recovery, or other downstream impacts.

Perimenopause also brings about a host of new nutrient needs that, if left unfulfilled, can lead to low energy availability for workouts, recovery, and physical progress. Moreover, the previously listed factors can impact not only the menstrual cycle and the individual's predisposition to certain conditions but can also impact the individual's perimenopausal and postmenopausal experience. It would therefore be useful for a woman's postmenopausal health to take actions during the perimenopausal stage to become healthier with eating, exercising, sleeping, and managing stress.

Existing systems that focus on the health needs of women typically focus on younger women. Such systems may do well predicting cycle length for the younger age group, predicting the fertile window, and other factors. Some other systems also exist that are capable of making general recommendations for a user in perimenopause. But these other systems typically give the same recommendation each month and during each of the four phases of the menstrual cycle. As a result, these existing systems treat a user in perimenopause the same as they would a pre-menopausal user by failing to adjust to a more erratic menstrual cycle or with a post-menopausal user having no cycle. In either case, the unique needs of individual users in this specific life phase are underserved by retrofitting recommendations to different life phases. In addition to failing to provide phase-specific recommendations, these existing solutions tend to rely on pre-built sets of recommendations and do not respond to the user's daily inputs or individualized health characteristics to refine outputs.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 shows an exemplary wearable device according to an embodiment;

FIG. 2 shows an exemplary analysis and recommendation system according to an embodiment;

FIG. 3 is a flow chart for an analysis and recommendation process according to an embodiment; and

FIG. 4 is a block diagram of an application for generating recommendations according to an embodiment.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, the subject technology is not limited to the specific details set forth herein and may be practiced using one or more implementations. In one or more instances, structures and components are shown in simplified form in order to avoid obscuring the concepts of the subject technology.

In the drawings referenced herein, like reference numerals designate identical or corresponding parts throughout the several views or embodiments.

According to an embodiment, the system collects data from users and presents that data after some processing has taken place. The system additionally personalizes recommendations to each user and continues to refine those recommendations based on user input and observational data from technology such as wearable devices, smart scales, and the like. Using a repository of possible sub-recommendations, the system can include machine learning that assembles these sub-recommendations into full, unique, daily recommendations for a variety of categories including, for example, food, exercise, sleep, and stress (FESS) management.

To generate the recommendations, the system uses historical menstrual data of a user (such as user-reported flow rate, menstrual period start date, menstrual symptoms, and the like), and collections of other users to predict future menstrual flow levels/rate as well as cycle phases of the user. Using both historical data from the user in conjunction with collections of data from other users enables the system to more accurately predict the menstrual cycle for users with less predictable cycles, like perimenopausal users. With more accurate predictions of the more variable cycles of perimenopausal users, the system can combine user inputs and other health data to make recommendations around FESS management appropriate to the menstrual phase to allow perimenopausal users to optimize their health through their behaviors. In addition, the system can combine user inputs and other health data to adjust recommendations according to needs and preferences.

To collect the health data, the system can gather health data from wearable technology (e.g., Fitbit, smart watch) and Internet of Things (IoT) technologies (e.g., scales, heart rate monitor, blood pressure monitor) that are capable of measuring and collecting relevant health data from a user. The collected health data, generally related to bodily function and characteristics, can include heartrate variability (HRV), basal body temperature (BBT), temperature fluctuations, resting heartrate (RHR), exercise heartrate, number of steps, weight, time asleep, and quality of sleep, as well as other relevant data. For BBT, the sensor can measure temperature directly or can measure a change from a set baseline temperature in lieu of actual temperature. In addition, the temperature can be measured by a wrist-worn, finger-worn, or other body-worn sensor, preferably configured to measure temperature continuously, or by an oral sensor or other sensor capable of measuring internal body temperature. The collected health data can be combined with user inputs to make health-related behavior recommendations that account for the user's changing menstrual cycle in perimenopause. To further individualize the recommendations, the system can use socioeconomic, geographical, and other data using a database of similar users and what has worked for them.

The system can be configured to learn from the user, both from user-entered information and from additional collected health data from wearable devices and IoT devices, to adjust its recommendations to help maximize health and fitness outcomes each day of the cycle by extrapolating the phase recommendations to an erratic cycle. The system can also respond to unique inputs that arise from perimenopausal symptoms or life events that can impair the user's ability to carry out the initial recommendations by giving a new recommendation suited to the specific user and wearable inputs. Further, the system can adjust future recommendations to enable the user to stay on track to meeting specific goals. By predicting future trends based on the data being collected, the system can forewarn the user with preparatory output, such as dietary strategies for mitigating cravings and supporting mental and physical health according to the predicted changing hormonal milieu.

The system can be configured to generate the recommendations by relying on a large volume and variety of data not available to an individual user to collect and utilize on their own. With this large volume and variety of data, it is possible to use machine learning to analyze the data, find clusters of similar data, and continually update and adjust findings based on newly collected data and responses. The findings and outputs from the machine learning can be used to improve, sharpen, and customize the FESS recommendations to the user according to that user's information and collected health data.

The system enables each user to go beyond simple static content and guide the user in a way that is tailored to each user's daily needs, saves them time, and allows them to have a plan that can change as their needs change. The system can make recommendations to allow the user to mitigate uncomfortable symptoms and improve their health through behavior modifications and can adjust these recommendations based on user feedback as well as input from devices' fitness goal setting, workout planning and tracking, food tracking, sleep improvement, stress management and menstrual cycle tracking. Beyond simplifying the number of applications that a user may need to employ to address each of these issues, the system also provides a specific technical improvement by using both user-entered information in combination with health data collected from the user through wearable devices and/or IoT devices, applying this data to determine future menstrual cycles and FESS recommendations, applying machine learning to the received data in combination with large volumes and varieties of data from a large collection of other users to determine similar data to refine and improve menstrual cycle determinations and FESS recommendations, and continually updating and adjusting the determinations and recommendations based on newly collected data and responses.

Using data from numerous users, applying computer-based algorithms including machine learning, and generating and repeatedly adjusting recommendations based on both user feedback and measurements of bodily functions through technical equipment such as electronic sensors, while still accounting for user inputs and health data, represents a technologically based improvement to existing systems. The system learns from the user and adjusts its recommendations to help maximize health and fitness outcomes each day of the menstrual cycle and can extrapolate the phase recommendations to an erratic menstrual cycle. Moreover, the system can respond to unique inputs that arise from perimenopausal symptoms or life events that can impair a user's ability to carry out the initial recommendations by giving a new recommendation suited to the specific user and device inputs. The system can likewise adjust future recommendations to enable the user to stay on track to meeting specific goals. By predicting future trends based on the data being collected, the system can forewarn the user with preparatory output, such as dietary strategies for mitigating cravings and supporting mental and physical health according to the predicted changing hormonal milieu.

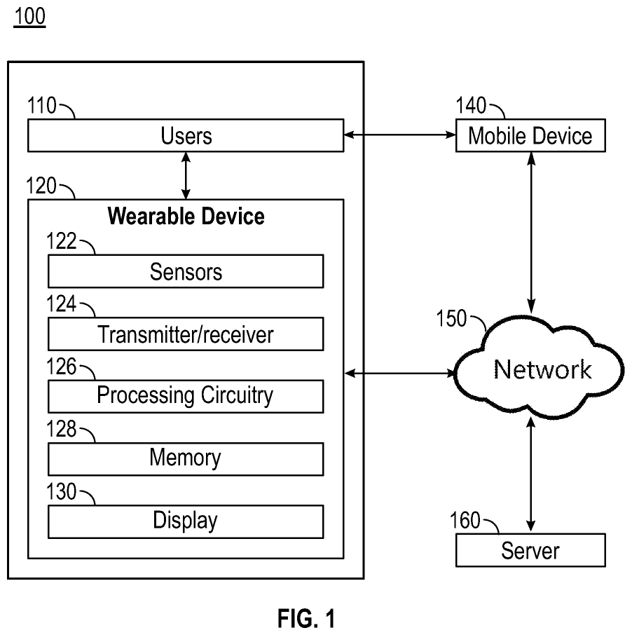

FIG. 1 is a block diagram of a system for analysis and recommendations according to an embodiment. As shown in FIG. 1, the system 100 includes a user 110, a wearable device 120, a mobile device 140, and a server 160. Although only one user 110 is shown, it should be understood that system 100 can include many users, hundreds, thousands, hundreds of thousands, or more. In addition, while each user 110 is a person capable of menstruating and is preferably perimenopausal, the system 100 can be also applied to users 110 that are premenopausal as well.

FIG. 1 further illustrates that the users 110 may interact with the wearable device 120 and/or the mobile device 140. The user may interact with the wearable device 120 actively using for example a user interface (e.g., display 130, optional control buttons (not shown)) of the wearable device 120. The user may also interact with the wearable device 120 passively for example through sensors that are integrated into the wearable device 120.

In addition, FIG. 1 illustrates that the user 110 may interact with the mobile device 140. For example, the user 110 may interact through a user interface (not shown) of the mobile device 140. Examples of user interfaces of the mobile device 140 include but are not limited to touchscreen, microphone, camera, vibration sensors, keyboard, mouse, control buttons, speakers, and/or the like.

FIG. 1 further illustrates that the wearable device 120, the mobile device 140, and the server 160 communicate over a network 150. In some cases, the wearable device 120 may communicate over the network 150 via a first wireless connection (e.g., personal area network (PAN)) to the mobile device 140 and use the mobile device 140 to communicate over the network 150 via a second wireless connection (e.g., local area network (LAN), wide area network (WAN), internet, or the like) to the server 160. However, the wearable device 120 may also communicate directly over the network 150 (e.g., peer-to-peer (P2P) network) to the server 160. Although examples are provided for wireless connection to the network 150, it is also conceivable for the wearable device 120 and/or the mobile device 140 to be connected to the network 150 using one or more wired connections (e.g., universal serial bus (USB), ethernet, high-definition multimedia interface (HDMI), fiber, or the like). The various connections and protocols used to communicate over the network 150 are described in greater detail below.

The wearable device 120 can include one or more sensors 122, a transmitter/receiver 124, processing circuitry 126, a memory 128, and a display 130. Wearable device 120 can be, for example, a wrist bracelet with sensors 122, transmitter/receiver 124, processing circuitry 126, memory 128, and display 130. Alternatively, wearable device 120 can be a wristwatch or smartwatch configured to include the same elements. Wearable device 120 can also be configured to be any other shape or form capable of being worn by a user, configured to include the elements shown in FIG. 1, and capable of being positioned so that the sensors 122 can properly measure one or more bodily functions.

Sensors 122 can be a chip, circuit, or sensor that is configured to detect bodily functions (i.e., health data) of a user when sensor 122 is, for example, next to or adjacent to a skin surface of the user. Sensor 122 is preferably configured to generate one or more electronic signals indicative of the detected bodily function. The bodily functions can include, for example, Heart Rate Variability (HRV), Resting Heart Rate (RHR), Exercise Minutes, Steps, Basal Body Temperature (BBT), Sleep, Random Eye Movement (REM) minutes, Non-Random Eye Movement (NREM) minutes, Sleep Quality, Weight, and Body Fat Percentage. In addition to sensors 122 in wearable device 120, sensors 122 can also be included in IoT devices like smart scales, heart rate monitors, and blood pressure monitors, for example.

Transmitter/receiver 124 can be, for example, an antenna and configured to transmit and receive signals and data to and from wearable device 120. The signals and data can be transmitted from transmitter 124 using communication standards as are known to one skilled in the art including, for example, Nearfield Communication (NFC), Bluetooth, WiFi, or cellular communication. When implemented with NFC or Bluetooth, the signals and data can be received and read when the wearable device 120 is proximate to a mobile device 140, such as a smart phone, mobile phone, tablet, or laptop, or proximate to a reader. When implemented with WiFi or cellular, the signals and data can be received and read from the wearable device 120 without the same distance limitations.

Processing circuitry 126 can include circuitry, such as a microprocessor, microcontroller, CPU, memory, RAM, and/or ROM, that can be configured to control all of the operations of wearable device 120 including controlling the operations of the body temperature sensor 102, transmitter 124, memory 128, and display 130. Processing circuitry 126 can also include GPS circuitry that generates location data according to the position of wearable device 120. Memory 128 can be a RAM, ROM, and/or other memory circuit capable of receiving, storing, and providing data and instructions that can be used by processing circuitry 126 to perform functions of wearable device 120. Although not shown in FIG. 1, wearable device 120 can also include the use and placement of a Radio Frequency Identification (RFID) chip, which can be used to allow or deny access to wearable device 120 as well as provide location data and a unique identifier for the wearable device 120 and/or user.

Display 130 can be, for example, an LCD or LED display that presents information and data to a user of wearable device 120. Display 130 can display information regarding bodily functions like HRV, RHR, exercise minutes, steps, BBT, sleep, REM minutes, NREM minutes, sleep quality, weight, and body fat percentage. Some or all of these measures could come from either a single device or from more than one device.

Wearable device 120 can be configured to capture data continuously, at specific times or periods, or in response to a request or user input. When capturing data continuously, for example, a user preferably has wearable device 120 nearby to a mobile device 140, such as a smartphone, that has an application linked to the operation of wearable device 120 that can store several hours or more of bodily function data from the user 110.

Although a mobile device 140 is used in examples disclosed herein, it is understood that any suitable electronic device that is capable of storing and/or transmitting to the server 160 (e.g., via the network 150) several hours or more of bodily function data from the user 110 may be used regardless of its intended mobility. For example, a man-machine interface (MMI) device capable of storing data and/or transmitting data via the network 150, for storage on the server 160, several hours or more of bodily function data from the user 110 may be used. Such MMI devices may actively interface with the user 110 and/or passively interact with the user 110. Examples of such suitable devices include but are not limited to: smart home devices (e.g., WiFi connected devices, TVs, displays, speakers, laptops, desktop computers, smart beds, refrigerators, thermostats, microwaves, ovens, or the like), smart office equipment (e.g., speaker phones, smart tables, conferencing equipment, security cameras, or the like), smart exercise equipment (e.g., tread mills, exercise bikes, sports equipment, weight lifting equipment, scales, or the like), and smart vehicles (e.g., automobiles, bicycles, motor bikes, boats, aircraft, autonomous drones, or the like).

Wearable device 120 is preferably configured to be waterproof such as for showering, pool usage, or spills, to enable continuous body temperature and HRV monitoring. Wearable device 120 is also preferably designed to be comfortable for extended wear, e.g., for 30 days, and can be formed with silicone or similar material that makes extended wear more comfortable as well as preferably being hypoallergenic and adjustable.

The wearable device 120 communicates over the network 150 with the mobile device 140 and/or the server 160 through various wireless and/or wired network devices (e.g., network servers, base stations, radio access networks (RAN), network routers, and the like). The wearable device 120 monitors and collects the bodily function data of the user 110 and supplies the bodily function data to the mobile device 140 and/or the server 160 via the network 150. Note that the communication protocol supported by the wearable device 120 is not particularly limited, and furthermore, the wearable device 120 can support a plurality of types of communication protocols.

The wearable device 120 performs wireless communication with the network 150 using one or more wireless protocols to connect and communicate with the mobile device 140 and/or the server 160. For example, the wearable device 120 may support wireless protocols such as ultra-low power (ULP), personal area networks (PAN), near field communication (NFC), and radio frequency identification (RFID) wireless protocols. Examples of these wireless protocols may include but are not limited to: Bluetooth Low Energy (BLE™), Bluetooth Smart™, ANT™, ANT+™, Topaz™, MiFARE™, MiFARE Ultralight™, ISO 14443, ISO 18092, logic link control protocol (LLCP), or the like). Furthermore, for example, the wearable device 120 may connect via a wired connection (not shown) to the mobile device or the server 160 via a connection terminal (not shown) (and a cable if necessary) by universal serial bus (USB), high-definition multimedia interface (HDMI™), mobile high-definition link (MIHL), or the like.

Moreover, for example, the wearable device 120 performs communication with a device (for example, application server or control server) existing on an external network (for example, the Internet, a cloud network, or a network peculiar to a business operator) via a base station or access point. Furthermore, for example, the wearable device 120 performs communication with a terminal existing near the user 110 (for example, pedestrian terminal or store terminal, or machine type communication (MTC) terminal) by using peer to peer (P2P) technology.

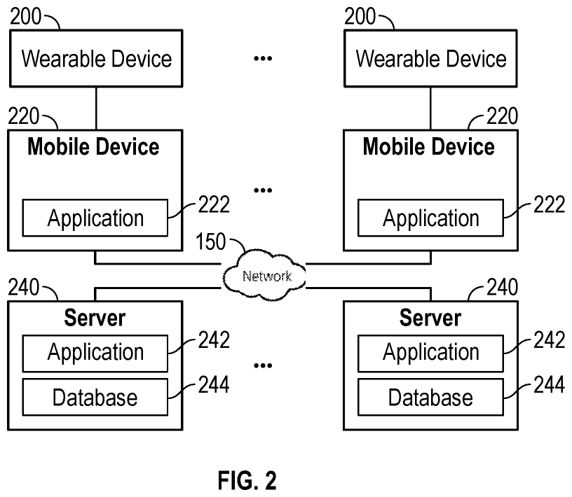

FIG. 2 shows an exemplary system comprising a plurality of the wearable device 110, a plurality of the mobile device 140, and a plurality of the server 160 according to an embodiment. As shown in FIG. 2, the system can include multiple wearable devices 200, multiple mobile devices 220, and multiple servers 240. Although multiple wearable devices 200, multiple mobile devices 220, and multiple servers 240 are shown in FIG. 2, it should be understood that the system can include just one of each instead of multiple ones of each. Wearable devices 200 in FIG. 2 can be configured in the same manner and have the same functionality as wearable device 120 in FIG. 1. Accordingly, further description of wearable device 200 is omitted.

Mobile device 220 can be a smartphone, mobile phone, tablet, laptop, or other mobile computing device capable of communicating with other devices and running applications. Mobile device 220 preferably includes processing circuitry and memory or storage configured to operate the mobile device, control the sending and receiving of data and signals, and running one or more applications including device application 222. Mobile device 220 can send and receive data from wearable device 200 using, for example, NFC, Bluetooth, WiFi, or cellular communication as explained above with regard to the mobile device 140.

Device application 222 can be a bidirectional tool that collects the data inputted by a user and bodily function data provided by one or more sensors in wearable device 200. Device application 222 can also collect bodily function data provided by other devices including by other applications in mobile device 220 and by IoT devices like smart scales, heart monitors, and blood pressure monitors. All of the bodily function data collected by device application 222 can be timestamped with the time and date of the collection along with other relevant data including an identifier for the user of wearable device 200 providing the bodily function data and a location of that user, thereby providing a real-time representation at the moment of collection. Device application 222 can be configured to control mobile device 220 to send the collected data to one or more of servers 240.

Server 240 can be a computing device capable of communicating with other devices and running applications. Server 240 preferably includes processing circuitry and memory, or storage configured to operate the server, control the sending and receiving of data and signals including over the Internet, through WiFi, and/or through cellular communication, and run one or more applications including server application 242. Server 240 can send and receive data from wearable device 200 and/or mobile device 220 using, for example, WiFi or cellular communication. Server 240 can be either a physical device, a virtual device, a service, or suite of services provided by one or more Cloud Service Providers, or other device such as the user's mobile device, that supplies the functionality needed by Device application 222 and/or Server application 242.

Server application 242 can be configured to be event driven, operate in real-time, and use database programming such as SQL, NoSQL or other database languages and protocols including optionally those used for bigdata applications such as Hadoop or other large-scale database applications. Server application 242 can also be configured with REST or representational state transfer or similar application programmer interface (API) to control data transfer between device application 222 and servers 240, as well as between servers 240 and a computing device operated by users. In operation, applications 242 in servers 240 can collect the bodily function data for each individual user of wearable device 200, along with other related data such as location, time, and user identifier, and store the data in one or more databases 244. All of the data can be provided into a contemporaneous data model for processing, such as by a machine learning algorithm.

The device application 222 can be downloaded by a user to the user's mobile device 220 from an application store associated with the mobile device 220. Alternatively, device application 222 can be configured to run on another computing device, such as a laptop, desktop PC, or other device capable of operating device application 222. The data input by the user includes static data, which can include user profile data and user preferences. With the device application 222 downloaded and operating, the user can begin to create a profile. The intake process for the profile includes information such as username, birth date, socioeconomic and biometric data, and application setup preferences. The device application 222 can also ask the user to provide the start date of their current menstrual cycle, plus the lengths of a number of prior cycles such as the last three cycles.

The device application 222 can also maintain a table of biological norms that can be referred to as a “cycle norms vector,” where a “vector” can be a “list of things” such as a spreadsheet or table of values used by the device application 222. The table of values can be configured to indicate what the typical hormone environment would be for a woman on a particular day of the menstrual cycle. This table provides the device application 222 with a way of beginning to predict appropriate recommendations for a specific user on a specific day, based on capacity and physiology resulting from specific nervous system activation. For example, on Day 12, estradiol is dominant and rising, progesterone is low, and luteinizing hormone is rising fast, which usually leads to a higher energy level and greater capacity to handle stress or challenging things like an intense workout. The parasympathetic nervous system is dominant on this day, and as such, stress hormones are optimal for higher capacity and workload. In contrast, on Day 25, progesterone is dominant, and both progesterone and estradiol are falling. While a user on this day is still capable of a challenging workout, a user may be better served (i.e., may recover better and avoid injury) by a different modality of exercise, such as yoga. Because the sympathetic nervous system is dominant during this part of the cycle, which results in elevated stress hormones and a reduced capacity for physical stress, such a different modality of exercise is more appropriate and effective.

The following tables list elements from a Cycle Norms Vector (CNV) that can be used by device application 222 and/or server application 242 for generating recommendations:

| Day | Estrogen | | Progesterone | | |
|---|---|---|---|---|---|
| of | Relative | Estrogen | Relative | Progesterone | Dominant |
| Cycle | Level | Direction | Level | Direction | Hormone |
| 1 | low | falling | low | falling | estrogen |
| 2 | low | falling | low | falling | estrogen |
| 3 | low | rising | low | falling | estrogen |
| 4 | low | rising | low | falling | estrogen |
| 5 | low | rising | low | falling | estrogen |
| 6 | low | rising | low | falling | estrogen |
| 7 | low | rising | low | rising | estrogen |
| 8 | low | rising | low | falling | estrogen |
| 9 | low | rising | low | rising | estrogen |
| 10 | med | rising | low | rising | estrogen |
| 11 | med | rising | low | falling | estrogen |
| 12 | high | rising | low | rising | estrogen |
| 13 | high | rising | low | rising | estrogen |
| 14 | high | falling | low | rising | estrogen |
| 15 | med | falling | low | rising | progesterone |
| 16 | low | falling | low | rising | progesterone |
| 17 | med | rising | med | rising | progesterone |
| 18 | med | rising | high | rising | progesterone |
| 19 | med | rising | high | rising | progesterone |
| 20 | med | rising | high | rising | progesterone |
| 21 | med | rising | high | rising | progesterone |
| 22 | med | falling | high | falling | progesterone |
| 23 | med | rising | high | falling | progesterone |
| 24 | med | falling | high | rising | progesterone |
| 25 | med | falling | med | falling | progesterone |
| 26 | med | falling | low | falling | progesterone |
| 27 | low | falling | low | falling | progesterone |
| 28 | low | falling | low | falling | progesterone |

| Day | | Mean | | | | Dominant | |
|---|---|---|---|---|---|---|---|
| of | Typical | BBT | Cycle | Ovarian | Uterine | Nervous System | |
| Cycle | Discharge | (F.) | Phase | Phase | Phase | (ANS) | Base Reaction |
| 1 | Eggwhite | 97.3 | Pre- | Follicular | Menstrual | Even | Even |
| | | | Ovulation | | | | |
| 2 | Eggwhite | 97.4 | Pre- | Follicular | Menstrual | Even | Even |
| | | | Ovulation | | | | |
| 3 | Creamy | 97.6 | Pre- | Follicular | Menstrual | Even | Even |
| | | | Ovulation | | | | |
| 4 | Creamy | 97.6 | Pre- | Follicular | Menstrual | Even | Even |
| | | | Ovulation | | | | |
| 5 | Eggwhite | 97.3 | Pre- | Follicular | Menstrual | Even | Even |
| | | | Ovulation | | | | |
| 6 | Creamy | 97.5 | Pre- | Follicular | Proliferative | Parasympathetic | RestAndDigest |
| | | | Ovulation | | | | |
| 7 | Creamy | 97.5 | Pre- | Follicular | Proliferative | Parasympathetic | RestAndDigest |
| | | | Ovulation | | | | |
| 8 | Creamy | 97.6 | Pre- | Follicular | Proliferative | Parasympathetic | RestAndDigest |
| | | | Ovulation | | | | |
| 9 | Eggwhite | 97.4 | Pre- | Follicular | Proliferative | Parasympathetic | RestAndDigest |
| | | | Ovulation | | | | |
| 10 | Creamy | 97.6 | Pre- | Follicular | Proliferative | Parasympathetic | RestAndDigest |
| | | | Ovulation | | | | |
| 11 | Creamy | 97.6 | Pre- | Follicular | Proliferative | Parasympathetic | RestAndDigest |
| | | | Ovulation | | | | |
| 12 | Creamy | 97.7 | Pre- | Follicular | Proliferative | Parasympathetic | RestAndDigest |
| | | | Ovulation | | | | |
| 13 | Eggwhite | 97.4 | Pre- | Follicular | Proliferative | Parasympathetic | RestAndDigest |
| | | | Ovulation | | | | |
| 14 | Creamy | 97.7 | Ovulation | Follicular | Proliferative | Parasympathetic | RestAndDigest |
| 15 | Creamy | 97.7 | Post- | Luteal | Secretory | Sympathetic | FightOrFlight |
| | | | Ovulation | | | | |
| 16 | Sticky | 98.3 | Post- | Luteal | Secretory | Sympathetic | FightOrFlight |
| | | | Ovulation | | | | |
| 17 | Sticky | 98.3 | Post- | Luteal | Secretory | Sympathetic | FightOrFlight |
| | | | Ovulation | | | | |
| 18 | None | 98.5 | Post- | Luteal | Secretory | Sympathetic | FightOrFlight |
| | | | Ovulation | | | | |
| 19 | None | 98.5 | Post- | Luteal | Secretory | Sympathetic | FightOrFlight |
| | | | Ovulation | | | | |
| 20 | Sticky | 98.1 | Post- | Luteal | Secretory | Sympathetic | FightOrFlight |
| | | | Ovulation | | | | |
| 21 | None | 98.5 | Post- | Luteal | Secretory | Sympathetic | FightOrFlight |
| | | | Ovulation | | | | |
| 22 | None | 98.5 | Post- | Luteal | Secretory | Sympathetic | FightOrFlight |
| | | | Ovulation | | | | |
| 23 | None | 98.3 | Post- | Luteal | Secretory | Sympathetic | FightOrFlight |
| | | | Ovulation | | | | |
| 24 | None | 98.5 | Post- | Luteal | Secretory | Sympathetic | FightOrFlight |
| | | | Ovulation | | | | |
| 25 | None | 98.4 | Post- | Luteal | Secretory | Sympathetic | FightOrFlight |
| | | | Ovulation | | | | |
| 26 | None | 98.5 | Post- | Luteal | Secretory | Sympathetic | FightOrFlight |
| | | | Ovulation | | | | |
| 27 | Sticky | 98.3 | Post- | Luteal | Secretory | Sympathetic | FightOrFlight |
| | | | Ovulation | | | | |
| 28 | Sticky | 98.3 | Post- | Luteal | Secretory | Sympathetic | FightOrFlight |
| | | | Ovulation | | | | |

This CNV chart provides a 28-day list, which is for a standard menstrual cycle, but the chart can be modified through a calculation to account for unusual or non-standard menstrual cycles, such as during perimenopause. To do so, a user's predicted cycle length is used to choose which “standard day” to use for the actual day's recommendation according to the following formula (1):

$$⌊ [ ( {D}_{u} - 1 ) * \frac{{L}_{s} - 1}{{L}_{u} - 1} ] + 1 ⌋ \qquad ( 1 )$$

where Du represents user cycle day, Ls represents standard cycle length, and Lu represents user cycle length. To implement in a spreadsheet program, equation (1) can be given by the formula: =ROUND((((Du−1)*(Ls−1)/(Lu−1))+1),0). For example, for a user with a 45-day predicted cycle, who is currently on day 20, device application 222 or server application 242 would use values from the CNV for Day 13. Similarly, for the same user on day 30, device application 222 or server application 242 would use values from CNV Day 19.

In addition to setting up the device application 222 with basic user profile data, the users can also be asked to provide some initial baseline data. One type of initial baseline data is a food vector. For the food vector, the user can be asked questions about what foods they have eaten over a certain period, such as the last four weeks or last month. Based on the responses, the device application 222 begins to populate the food vector with a historic list of food types, quantities, and preferences.

Another type of initial baseline data is an exercise vector. For the exercise vector, the user is asked questions about what types of exercises they have done over a certain period, such as the last four weeks or last month. Based on the responses, the device application 222 begins to populate the exercise vectors with a historic list of exercise modalities, workout frequency, equipment availability, user ability and skill level, preferences, and any limitations the user may have for which the device application 222 needs to know.

For a sleep vector, the user is asked questions about how they have been sleeping over a certain period, such as the last four weeks or last month. Based on the responses, the device application 222 begins to populate the sleep vector with a historic list of sleep patterns, what the user does (if anything) to prepare for a good night's sleep, waking alertness, waking energy level, daytime sleepiness, and habits known to help or interrupt sleep.

For a stress management vector, the user is asked questions about their stress levels over a certain period, such as the last four weeks or last month, and what they do to manage their stress. Based on the response, the device application 222 begins to populate the stress management vector with a historic list of stress patterns, as well as user stress management knowledge, skills, interests, and things the user has been using to manage their stress.

Finally, the user is also requested to provide baseline data about symptoms. Here, the user is asked to input information about menstrual and perimenopause symptoms the user has experienced over a certain period, such as the last four weeks or a certain number of menstrual cycles. Based on the responses, the device application 222 begins to populate a symptoms log with a list of symptoms the user has experienced so that the device application 222 can take these symptoms into account to help the user make changes to improve their health and wellbeing.

The following tablesshowexamplesofthetypesofuser-inputteddatathatcanberequested for the user profile data and user baseline data:

User Profile and Other User-Provided Data (Example of User Input in Right Column Below)

| | |
|---|---|
| First Name | Sarah |
| Last Name | Smith |
| Email | ssmith@nomail.com |
| Birthdate | Jan. 1, 1971 |
| City, ST, ZIP | Austin, TX 78716 |
| Gender Identity | Woman |
| Biological Sex | Female |
| Race | White |
| Ethnicity | Non-Hispanic |
| HRT (MHT) Status (Hormone | Estradiol/Testosterone/DHEA in |
| Replacement Therapy) or Menopausal | compounded cream, Progesterone in pill |
| Hormone Therapy) | |
| First Day of current cycle (date) | Dec. 11, 2021 |
| Lengths of last 3 cycles, if known | [38, 35, 46] |
| Activity Level (1-5)* | 3 |
| Height (ft, in) | 5′ 2″ (64 in) |
| Neck (in) | 14 |
| Waist (in) | 30 |
| Hips (in) | 43 |
| Type of diet preferred | omnivore |

TABLE 1
Activity Levels

| Activity Factor | Rank | Factor** |
|---|---|---|
| Sedentary | 1 | 1.2 |
| Light Activity | 2 | 1.375 |
| Moderate Activity | 3 | 1.55 |
| Very Active | 4 | 1.725 |
| Extra Active | 5 | 1.9 |

**Multiply BMR × Activity Factor to get Maintenance Calories Maintenance Calories = Calories to remain at current weight

Goals (Example of User Input in Right Column Below)

| | |
|---|---|
| Weight (y/n) | Y |
| Lbs/wk gain/loss | −1 |
| Symptom Reduction | Y |
| Symptoms to focus on | [brain fog, hot flashes] |
| Improve Sleep | Y |
| Reduce Stress | N |

User Preferences (Example of User Input in Right Column Below)

| | |
|---|---|
| Temperature Units (F/C) | F |
| Weight Units (Lbs/kg) | lbs |
| Exercise equipment available | [Barbells, dumbbells, kettlebells, bands, |
| | bodyweight, medicine/slam balls, treadmill, |
| | elliptical, rower, assault bike, stationary |
| | bike] |
| Preferred exercise modality(ies) | [Strength training, rower, spin, yoga, |
| | dancel |
| Max Number of Days/Wk Available to | 3 |
| Exercise | |
| Days available for exercise | Monday, Wednesday, Friday, Saturday] |
| Physical limitations | Some foot pain (joints-big toe, ankle), hip |
| | and lower back pain, tennis elbow |
| Dietary limitations/allergies/sensitivities | [Corn, soy, dairy, wheat] |

What Symptoms would You Like to Track? (Selected Symptoms are in Bold Type)

| | |
|---|---|
| Allergies | Anxiety |
| Back Pain | Bloating |
| Body Odor | Brain Fog |
| Breast Pain | Brittle Nails |
| Burning Tongue | Cravings |
| Constipation | Depression |
| Diarrhea | Difficulty Concentrating |
| Digestive Problems | Dizziness |
| Early Waking | Electric Shocks |
| Fatigue | Gum Problems |
| Hair Loss | Headaches |
| Hot Flashes | Incontinence |
| Insomnia | Irregular Heartbeat |
| Irregular Periods | Irritability |
| Itchy Skin | Joint Pain |
| Loss of Libido | Memory Lapses |
| Mood Swings | Muscle Tension |
| Night Sweats | Osteoporosis |
| Panic Disorder | Painful Sex |
| Restless Leg | Sleep Disorders |
| Tingling Extremities | Vaginal Dryness |
| Weight Gain | |

Do You Regularly Consume the Following? (Selected Items are in Bold Type)

| Fruit | Vegetables |
|---|---|
| Animal-based protein (including eggs | Vegetable-based protein (including |
| and dairy) | beans/legumes, soy products, quinoa) |
| Fish | Healthy fats (nuts, seeds, oils, avocado, |
| | dairy products, fish oil, fatty fish) |
| Grains (cereals, rice, products made | Starchy vegetables (squash, potatoes, sweet |
| from them) | potatoes, tubers) |
| Caffeine | Alcohol |
| Added sugar | Sugar-sweetened beverages (including fruit |
| | juices) |
| Processed foods/food products | Artificial sweeteners |
| Fast food (more than 2/month) | Cigarettes/cannabis |
| Restaurant meals (more than 2/month) | Opioids, prescription or otherwise |
| Over-the-counter medications. List? | Over-the-counter vitamins/supplements. |
| | List? |

Baseline User Inputs:

Food: What has Your Food Intake Looked Like Over the Last 4 Weeks? (Example of User Input in Right Column Below)

| | |
|---|---|
| How many meals and snacks do you eat | 4 |
| each day? | |
| How many times each week do you eat the | Breakfast: 0 |
| following meals away from home? | Lunch: 5 |
| | Dinner: 3 |
| What types of eating places do you | Fast Food: Y |
| frequently visit? | Diner/Cafeteria: N |
| | Restaurant: Y |
| | Other: N |
| On average, how many pieces of fruit do | 1 |
| you eat each day? | |
| On average, how many servings of | 2 |
| vegetables do you eat each day? | |
| On average, how many times a week do you | 7 |
| eat whole grains? | |
| How many times a week do you eat red | 2 |
| meat? | |
| How many times a week do you eat chicken | 3 |
| or turkey? | |
| How many times a week do you eat fish or | 2 |
| shellfish? | |
| How many times a week do you eat fatty | 2 |
| cold-water fish? | |
| How many times a week do you eat desserts | 12 |
| or sweets? | |
| How many times a week do you eat refined | 7 |
| carbohydrates (i.e., white bread, boxed | |
| cereal, baked goods) | |
| How many days per week do you drink | 5 |
| alcohol? | |
| How many drinks per day do you have | 2 |
| when you do drink? | |
| On average, how many servings of caffeine | 3 |
| do you have each day? | |

Exercise: What has Your Exercise Looked Like Over the Last 4 Weeks? (Example of User Input in Right Column Below)

| | |
|---|---|
| Cardiovascular | Type 1: Walking |
| | Times/week? 5 |
| | Duration/steps? ~8500 steps |
| | Intensity (1 = light, 4 = vigorous) 1 |
| | Type 2: HIIT |
| | Times/week? 1 |
| | Duration? 45 minutes |
| | Intensity? 4 |
| | Type 3: Jump training |
| | Times/week? 1 |
| | Duration? 15 minutes |
| | Intensity? 4 |
| Strength training | Intensity? 4 |
| | What type? Weightlifting-bodybuilding |
| | Times/week? 2-3 |
| | Duration? 60 minutes |
| Flexibility/mobility/Balance | What type? Yoga |
| | Times/week? 2-3 |
| | Duration? 30 minutes |

Sleep: Over the Last 4 Weeks, how have You been Sleeping? (Example of User Input in Right Column Below)

| | |
|---|---|
| How many hours, on average, do you | 7 hrs |
| sleep? | |
| Rate your typical sleep quality | [Poor, Acceptable, Average, Good, Great] |
| | Average |
| How is your energy, on average? | [Poor, Acceptable, Average, Good, Great] |
| | Acceptable |
| Do you take medication to help you sleep? | N |
| Do you take over the counter supplements | Y |
| to help you sleep? | |
| What time do you begin to wind down for | 9:30 |
| bed each night? | |
| Do you avoid work-related reading 2 hours | N |
| before bedtime? | |

Stress Management: Over the Last Month, which of the Following have You Done Regularly? (Example User Input in Right Column Below)

| | |
|---|---|
| Breathing Exercises | Y |
| Meditation | N |
| Yoga | Y |
| Slow Walks | N |
| Exercise | Y |
| Social Conversation | Y |
| Self-Care | Y |

In addition to the user-inputted data, the device application 222 can collect bodily function or health data from the user. The bodily function data can be collected from the wearable device 200, from the mobile device 220, and/or from an IoT device associated with the user. To enable this collection, the device application 222 requests the user to allow the device application 222 access to the bodily function data on their wearable device 200, mobile device 220, and/or IoT device. Depending on the device, this data will include such fields as: Heart Rate Variability (HRV), Resting Heart Rate (RHR), Exercise Minutes, Steps, Basal Body Temperature (BBT), Sleep [Random Eye Movement (REM) minutes, Non-Random Eye Movement (NREM) minutes], Sleep Quality, Weight, and Body Fat Percentage.

Heart rate variability (HRV) is a cycle regulated by the hypothalamus, and HRV is an indication of the health of the nervous system. To determine or detect HRV, the wearable device 220 can include a pulse sensor that measures the time between heartbeats. Increased variability in time intervals is an indication of the body's ability to respond quickly to stimuli, whereas decreased variability indicates the converse. HRV can be affected by sleep, by alcohol intake, caffeine, nutrition, illness, hydration, and/or exercise.

Basal body temperature (BBT) is a user's temperature when the user wakes up in the morning. As detailed in the previous CNV chart, BBT is naturally elevated at ovulation, and remains slightly elevated through the end of the cycle. Additionally, real-time body temperature can be detected and monitored to detect unusual body temperature changes that may correspond, for example, to the user experiencing hot flashes. Hot flashes can occur in perimenopausal users as a result of the hypothalamus' impaired ability to regulate core body temperature. Essentially, decreased estrogen reduces the range of tolerable core temperature fluctuation; when the hypothalamus perceives the core body temperature is too high, it will disperse heat through the extremities. This results in a quick change in distal body temperature even when the user is not exercising or otherwise performing some activity requiring a high level of exertion. There exists a correlation between hot flashes and HRV, in that reduced HRV has been observed in some women with increased body mass index (BMI) and severe hot flashes. Accordingly, since activities like exercise can improve HRV, it is likely that recommendations to improve HRV can correspondingly reduce hot flashes in the user.

The device application 222 can also take the user-inputted data and collected bodily function data and calculate values including HRV trends, User Cycle Day (based on standard cycle adjusted for this user's actual cycle length), Next Cycle Start Date, Sleep Debt, User Average Cycle Length, Menstrual Symptom Trends, Perimenopause Symptom Trends, and Capacity Score. Capacity Score (CS) is defined as a user's ability to do work or take on stress. For the user, this means that a high CS correlates to the ability to benefit from an intense workout, more easily deal with life or work stresses, and generally enjoy peak performance. A low CS correlates with the likelihood of benefiting more from a restorative workout, and the advisability of choosing less stressful activities

CS can vary from day to day and week to week during the menstrual cycle. The device application 222 can make recommendations to help maximize CS for a particular day and will reveal trends and correlate certain activities with the resulting CS so that the user can make informed decisions that can lead to better outcomes. CS is a function of several variables including wearable data such as HRV, Sleep Quantity, user-inputted variables such as Waking Energy Level and symptom quantity/severity, and Cycle Standard Hormone Levels. This is an improvement over existing systems that rely solely on wearable data to calculate a readiness score, as it can more fully reflect the user's readiness at any given moment. Of these factors, HRV plays a significant role in determining day to day differences, as well as departures from cycle norms. Higher HRV would lead to a higher CS and therefore greater ability to perform, whereas lower HRV is an indicator that the body is dealing with extra stress, and the device application 222 will make recommendations accordingly.

The following tables show examples of the types of collected health data (i.e., bodily function data) for which a user can be requested to provide access and enable detection and/or derivation of health data for the user:

Device Data (Ask the User to Allow Access)

| Heart Rate Variability (HRV) | 31 ms |
|---|---|
| Weight (lbs) | 140 |
| Body Fat % | 39 |

Calculated Data

| | |
|---|---|
| Age | 51 |
| BMI | 25.6 |
| Body Fat % | 39% |

Baseline Wearable/IoT Inputs

| | |
|---|---|
| HRV | (all same as above during intake for |
| | baseline) |
| RHR | |
| BBT | |
| Steps | |
| Weight | |

Derived Inputs

| | |
|---|---|
| HRV (current) | (data derived from inputs above) |
| HRV (7-day trend) | |
| CS (current day) | |
| CS (7-day trend) | |
| Sleep Debt (current) | |
| Avg Cycle Length for This User | |
| This user cycle length to 28-day standard conversion | |
| User's Cycle Day (what day of their cycle are they | |
| on?) | |
| User's Cycle Day Standardized (converted to 28-day | |
| range) | |

Once the baseline data (including user profile data, other relevant user-inputted data, and bodily function data) has been collected, the device application 222 can begin daily data gathering. To do so, the user can be prompted to log information into the device application 222 at regular times and intervals. These logs can include:

Food: For food, the device application 222 can prompt the user to log information about the foods they ate since the last log entry, as well as any cravings they had, how they liked their foods, whether they followed the device application 222 recommendations, and how they liked those recommendations.

Exercise: For exercise, the device application 222 can prompt the user to log information about the types of exercise performed since the last log, whether they followed the plan recommended by the device application 222, and a rating on how they liked that workout.

Sleep: For sleep, the device application 222 can prompt the user to log information about how they slept the night before, as well as their energy level and alertness. The device application 222 also prompts the user to log whether they followed the previous sleep recommendations, and how they liked those recommendations.

Stress Management: For stress management, the device application 222 can prompt the user to log information about their stress levels and types since the last log entry, as well as whether they followed the stress management guidance and how they liked that guidance.

In addition to the user-inputted data, additional bodily function data can be collected from the devices. To do so, the device application 222 can collect data from the user's wearable device 200, mobile device 222, and/or IoT devices throughout the day and night. This data includes, for example, HRV, BBT, Sleep, Steps, and Weight.

The following tables show examples of the types of user-inputted data and bodily function data that can be collected each day by the device application 222.

Daily User Inputs:

Food (Logged in the PM)

| | |
|---|---|
| How did you eat today? | |
| | I ate to fuel my body and mind |
| | I ate with friends/family |
| | I ate to satisfy cravings |
| | I ate for my emotions |
| What did you crave today? | |
| | Chocolate |
| | Salty snacks |
| | Sweets/carbs |
| | Alcohol |
| How did it make you feel? | |
| | My choices made me feel great |
| | My choices made me feel blah |
| | My choices made me feel sluggish |
| | My choices made me feel hungry/underfed |

Exercise (Logged in the PM or During the Day)

| | |
|---|---|
| Strength | [1, 2, 3, 4] |
| Conditioning | [1, 2, 3, 4] |
| Flexibility/Balance | [1, 2, 3, 4] |
| How was it? | [1, 2, 3, 4, 5] |

(The user can be prompted to indicate how much of their exercise for the day was associated with each exercise type. This will let the device application 222 determine whether the user is missing key exercise types in their routine or if they have been following a balanced plan.)

Sleep (Logged in the AM)

| | |
|---|---|
| How many hours did you sleep? | 5.5 |
| How do you feel today? | [Exhausted, Okay, Good, Great] |

Stress Management (Logged in the PM)

| | |
|---|---|
| What did you do to manage stress today? | € Breathing exercises/meditation |
| | € Exercise |
| | € Yoga |
| | € Glass of wine |
| | € Other |
| How helpful was it? | [Not Helpful, Somewhat Helpful, Helpful, |
| | Very Helpful] |

Feedback Inputs—Wearable/IoT (Logged Continuously)

HRV
RHR
BBT
Sleep
Steps
Weight

After the device application 222 collects the daily logs from the user as well as the daily input from the devices, it uses that information to refine recommendations for the following day/week. Weekly and monthly plans can change daily, analogously to weather forecast changes, based on inputs from the previous day. The user can be presented each morning with that day's plan, as well as an updated outlook for the week and month. The pattern is repeated each day with the user providing feedback and the system generating increasingly accurate recommendations for that user.

As more users use server application 242, the system can use similarity data to accelerate its arrival at the optimal set of recommendations for each user. This acceleration can be implemented, for example, by implementing a machine learning algorithm in server application 242 operating on one or more servers 240. One example of a machine learning algorithm that can be implemented in server application 242 is a kNN index: kNN (k-Nearest Neighbor), which is a machine learning algorithm used to assess a new data point based on similarities with existing data points, and make predictions using that data.

Server application 242 can also include a machine learning (ML)-based recommendation system that takes the user-inputted data along with other data sources including the bodily function data from wearable device 200, mobile device 220, and/or IoT devices and generates recommendations tailored to the user for the areas of Food, Exercise, Sleep and Stress Management (FESS). This ML-based recommendation system can be referred to as a FESS engine. The FESS engine generates the recommendations based on the user's history and trends for each of these areas (i.e., food, exercise, sleep, stress management), plus the user's hormone environment (e.g., based on the phase of the user's menstrual cycle), preferences and abilities. Like the kNN index ML-algorithm, the FESS engine also makes use of data regarding similar users in order to refine the recommendations. The FESS engine internally uses a neural network to make predictions for what will benefit the user the most during the time period covered by the recommendation, for instance, a day or a week, as well as what the user will likely accept (i.e., likely that the user will perform the recommended plan) and rate highly. The FESS engine can be configured to store all its recommendations as well as user ratings and measured outcomes and uses this data to predict from what FESS recommendations a specific user might benefit.

To facilitate the operation of the FESS engine, server application 242 can include a list of content items that the recommender system can use to assemble for food, exercise, sleep, and stress management recommendations. For food recommendations, server application 242 can maintain information about different foods and how they may relate to specific user situations. For exercise recommendations, server application 242 can maintain a large list of exercises that are arranged by modality (i.e.: weight training, yoga, running), as well as equipment needed and the category of exercise covered by the item, including Strength, Conditioning, Balance, and Flexibility. For sleep recommendations, server application 242 can maintain items that are designed to help a user better prepare for a good night's sleep, and each item has attributes to help the system choose recommendations that would be helpful for a particular part of the menstrual cycle or that would be appropriate based on a particular range of CS. And for stress management recommendations, server application 242 can maintain items such as meditation and breathing exercises, and each item has attributes to help the system choose recommendations that would be helpful for a particular part of the menstrual cycle or that would be appropriate based on a particular range of CS.

In addition to FESS recommendations, server application 242 can also provide educational content for users. For example, server application 242 can contain a list of content items designed to help the user gain a better understanding of the menstrual cycle, as well as general women's health and fitness topics, information about perimenopause, menopause, and other topics that may help the user put the recommendations into context.

Using both the FESS engine and the kNN index ML-algorithm, server application 242 can generate an initial set of recommendations after the user sets up device application 222 and provides the requested initial information. The initial set of recommendations is based on the profile data, other user-inputted data (e.g., user preferences, symptoms, food, exercise, sleep, stress management), bodily function data from wearable device 200, mobile device 220, and/or IoT devices, derived inputs based on the collected bodily function data (e.g., HRV, HRV trend, CS, CS trend, sleep debt, average cycle length, user's cycle data), the FESS engine, and the kNN index. Server 240 can send the recommendations generated by server application 242 to mobile device 220, which provides the recommendations to device application 222 to present the recommendations to the user with requests for the user to follow the plan according to the recommendations.

After receiving and implementing the plan based on the FESS recommendations generated by server application 242 and presented through device application 222, the user is given an opportunity to follow the plan and is subsequently prompted by device application 222 to provide feedback and logs. The feedback and logs can be used by server application 242 to refine the recommendations for the next time period. For example, once the user has followed the plan and provided good ratings for a certain period, such as seven consecutive days, server application 242 can generate updated recommendations based on more than just the data collected during intake, but rather by a continuous loop that also takes into account the feedback and logs, CS, the FESS engine, the kNN index, and the cycle norms vector.

Plans produced by server application 242 can appear internally as a table of values indicating what type of recommendation will appear. From there, a selection is made from the list of modular content based on a user rating prediction. For each element of the plan, two steps are taken based on the available data, as described above:
1. Predict what set of items will benefit the user the most over the time window being recommended.
2. From the set of items generated in (1), predict what subset the user will actually follow and subsequently rate highly.

Plans can be presented to the user in a timeline format, so they can be referred to after the dates have passed, to help with planning. An exemplary user plan can include the following, where one day's recommendations for each of eight phases is listed, along with adjustments to accommodate the unique (sample) fluctuations of the user's CS:

P1 (Days 1-2—Menses at Beginning of Cycle):

Food: In this phase, aim for a macronutrient breakdown of 40% protein, 30% carbohydrates, and 30% fats. Plan to eat 0.7-1 gram of protein and 0.5 gram of fat for each pound of bodyweight. Eat more iron-rich food to support the user through their period. Good iron sources are lean poultry, fish, and eggs. The body readily absorbs the iron in these foods. Plant-based iron sources include dark green leafy vegetables. The body will absorb more iron from plant foods when consuming them with vitamin C sources, such as oranges, tomatoes, and red/yellow/orange peppers.

Exercise: It is preferable to prioritize rest and recovery during P1. While this could normally mean a mid-day nap to help make up for lost sleep from the previous night, you logged over 7 hours of sleep and your HRV is higher than usual. Bonus! That means you can benefit from a moderate workout today—moderate being key. In other phases, the best stress reducer can be an intense workout, but in P1—even with a higher than usual CS—you should reduce the intensity of the workout. If you choose to work out, instead of big barbell complexes, focus more on single-side dumbbell movements that challenge balance.

Sleep: Make a list of the things that need to get done tomorrow. This helps to get it out of mind. Often trying to keep to-do items catalogued in mind sets off anxious thought spirals that conspire to keep you awake.

Stress: Practice one minute of box breathing as a meditation: Breathe in while counting to 4, then hold for a count of 4. Then exhale while counting to 4 and hold again for a count of 4. Intentional breathing exercises such as this increase presence, mindfulness, and calm while allowing mind and body to relax for recovery.

P2 (Days 3-5—Resolve of Menses, Low Hormone Phase):

Food: Eat fish such as salmon during this phase. The omega-3 fats in salmon fight inflammation, which is important as the period resolves in phase 2. It also supports better mood. While fatty fish is important every week of the cycle, it is needed even more during this phase.

Exercise: Energy climbs with estrogen at the beginning of Phase 2, but you should still avoid high-impact activities for the next few days until sufficient protective hormones circulate to safeguard ligaments and tendons. Activities like cycling, rowing, and strength training are great this week, but leave running and jumping for the next phase. Try to keep the intensity level moderate in all workouts this week. Don't worry, you'll be stepping on the gas in the next phase!

Sleep: Take a relaxing walk after dinner, at least 3 hours before bed. A casual stroll after dinner supports good digestion and circulation. Curtailing eating 3 hours before bed will allow the body to finish digesting first to enable deeper, restorative sleep.

Stress: Try to schedule a massage. Any activity that reduces stress on the body and mind will be beneficial to recovery.

P3 (Days 6-9):

Food: In this phase, aim for a macronutrient breakdown of 40% protein, 40% carbohydrates, and 20% fats. The added carbohydrate supports increasing work capacity and the ability to handle more intensity in the gym. Choose plant-based proteins from whole-food sources once a day this phase. Plant-based proteins are shown to help women manage their hormonal symptoms but carry more carbohydrate than animal-based foods do. Complete plant-based proteins include edamame and quinoa. Beans, legumes, nuts, and seeds are additional good sources of plant-based proteins but should be combined with a grain.

Exercise: Phase 3 is for crushing it! Estrogen is peaking and you probably feel on top of the world. Feeling unstoppable? Take that energy into the gym! Heavy compound exercises like squats and deadlifts, pullups (assisted or not), and bench press are in order this week. Keep reps low and weights high. Allow yourself to express energy! Estrogen is an anabolic hormone. And even if it's erratic now, excellent muscle tissue can be built with heavy lifting in this phase.

Sleep: Get about 30 minutes of sun exposure before 10 am. Exposing skin to early sun allows the body to synthesize vitamin D and sunlight exposure to your eyes causes serotonin synthesis in the brain, which is subsequently converted to melatonin. It's true! A good night's sleep starts in the morning.

Stress: Take a hot bath with magnesium salts. Magnesium soothes tired muscles and relaxes a tense mind. Additionally, a hot bath 30-60 minutes before bed can enhance deep sleep.

P4 (Days 10-13):

Food: Add a serving of starchy carbohydrate to dinner. The starch calms the central nervous system and makes it easier to wind down for sleep. Choose foods like sweet potatoes and winter squashes for added fiber.

Exercise: Though you may be feeling amped mentally and physically right now, your HRV is trending lower than usual. If you're stressed, let playfulness guide you to a more gratifying workout today. What sounds fun? Follow that to what could be a new workout muse! Things to try: kickboxing, playground parkour, dance lessons, animal flow, a new team sport. New equipment like macebells or Indian clubs may be fun and pique interest. Let curiosity be your guide to a new love!

Sleep: Spend 10 minutes in easy yoga stretching 30 minutes before bed. The deep breathing will calm the nervous system and may improve HRV. The gentle stretches will relax muscles for deep sleep, which is needed based on the lower HRV.

Stress: Planning the work schedule around the phases of your cycle can relieve stress and increase success. Estrogen is peaking this week, making you feel in charge. You may also notice more articulate speech and faster thinking. Time to crush that presentation!

P5 (Days 14-16, Ovulation):

Food: Quality sleep may be more difficult after ovulating in this phase, but there are foods that can help. Case in point: Drink tart cherry juice before bed. It is the only food source of naturally occurring melatonin, and has very little sugar, so it leads to minimal insulin response. Mix about ½ cup with sparkling water and pour over ice.

Exercise: In phase 5, exercise capacity is still robust, so a high workload remains sustainable. Plan heavy lifting these next few days. Start the week with a full-body barbell workout that includes compound exercises like front squats, overhead press, deadlifts, bent over row, and bench press. Keep reps low and weight load high. And don't forget to strengthen the core—the powerhouse! Add planks to each workout this week.

Sleep: Avoid caffeine for at least 6 hours before sleep. It takes 6 hours for one cup of coffee to clear receptors in the brain enough to let you ease off to sleep. That afternoon pick-me-up risks residual caffeine keeping you up at night. Added benefits of reducing caffeine intake include calming an overactive bladder and reducing hot flashes.

Stress: Insomnia can lead to sleep-specific anxiety. Interrupting the thought spiral with grace before it can begin is the best remedy. If awake in the middle of the night, let yourself know it is okay to just rest your body. Spend some time reflecting on gratitude and calming the body and mind with deep breathing.

P6 (Days 17-20):

Food: The second half of the cycle makes one more insulin resistant. For that reason, aim for a macronutrient breakdown of 40% protein, 30% carbohydrate, and 30% fat. Choose complex carbs only, and only around workouts, when the body is primed to absorb and use them. At other times, fill up on protein, vegetables, and healthy fats.

Exercise: As estrogen drops and progesterone rises, work capacity is slowly decreasing as well. Moderate intensity in workouts remains needed because it will help increase insulin sensitivity. Now is a good time to use dumbbells and kettlebells and to shoot for 60% of max in all lifts using a 6-10 rep range. Make time to focus on mobility and stretching.

Sleep: Deep sleep may be more challenging during this phase. Here is a sleep ritual to try this week: An hour before bed, take a hot shower. Then allow the body to cool before getting under the covers. Increasing core body temperature and allowing it to come down fosters deep sleep.

Stress: Nurturing deep connections in relationships has been shown to reduce cortisol. Studies also show that feelings of inclusion—knowing you are not alone—help women have a more positive outlook through perimenopause. Conversely, isolation can deepen negative feelings toward the transition being experienced. Therefore, time connecting with friends is vital to mental health and will strengthen mental, physiological, and spiritual well-being. Girls' night out?

P7 (Days 21-23):

Food: Estrogen is cresting this week, which can make you feel perfectly settled in the driver's seat! Having control without being controlled. Let's keep that rolling; when estrogen declines, and progesterone begins to climb, this hormone change can trigger junk food cravings. Eat certain foods now to keep those cravings at bay next phase. Make sure to have avocados, citrus, olives, and other fruits and vegetables rich in healthy fats and antioxidants on hand to fortify resolve and nutrient stores.

Exercise: Take extra care with mobility and flexibility. The achiness and added fatigue that come with this phase are best dealt with by prioritizing mobility and self-myofascial release, such as trigger therapy with a ball or foam rolling. Add a yoga class as well to benefit from deep, restorative stretching and balance training.

Sleep: Sleep is always important for health but can be particularly difficult to get in this phase. Pay attention to your sleep environment to help encourage good sleep. Make sure the room is dark and cool. Even minimal light can disturb light sleep. The optimal temperature for sleep is <68 F. Keeping the room cool also brings the added benefit of fighting night sweats.

Stress: Face-to-face time may drain your energy right now. You may find that you thrive more focusing on solitary tasks. This phase is a good time to close the office door and complete those projects that require head-down attention. Working with the cycle can turn work from a grind to a flow, which will inevitably reduce stress and help create the life of dreams.

P8 (Days 24-28—Potential PMS):

Food: Since water retention may be occurring, minimize salt to combat it. Water retention is the body's way to balance blood pressure, which is naturally low at this time of the month, and even more so during perimenopause. The less salty foods eaten, the less water the body will need to hold onto. Also, pushing fluids by consuming more water will help combat cycle-related bloat. The less water, the more concentrated the blood gets, forcing the body to retain as much water as possible to dilute it. Water can also be obtained from foods. Crisp vegetables like cucumbers and fruits like melons are excellent hydration sources.

Exercise: All hormones are dropping in this phase, which signals the start of the period. With declining hormones comes impaired recovery and reduced capacity. Your HRV and sleep are way down this week, too. Those are all signs that exercise should be light and restorative today. Walking, yoga, and core work are good today. Pay extra attention to mobility for recovery. Don't worry about slacking; this sets up for gains throughout the next cycle.

Sleep: The body needs more recovery support during this phase than any other, and you seem to be having trouble getting enough sleep. Try to extend the time available for sleep by going to bed an hour early tonight or clearing the morning calendar to get an extra hour in bed tomorrow.

Stress: If you suffer from PMS, stress can cause mood swings and mental anguish during this phase. Increased recovery makes you more resilient to the stress that tends to percolate and overwhelm. Spending time on a gratitude practice and ensuring adequate sleep can go a long way.

The following exemplary scenarios help illustrate how a user may use server application 242 to generate and refine FESS recommendations. These scenarios depict how different inputs from the user and wearable devices/mobile devices/IoT devices change the recommendation.

In common to each exemplary scenario and as explained above, the user first downloads the device application 222 and answers a variety of intake questions to ascertain the starting point, cycle history, fitness experience and level, and other relevant data. In each of the following exemplary scenarios, the user is assumed to be experienced in multi-modal functional fitness workouts, which use a variety of high-intensity modalities, has access to free weights, and is experienced with regular use, as well as rowing, running, and cycling for cardiovascular work. The user's goals with device application 222 are to lose some stubborn belly fat, regulate her energy levels and cravings, resolve stress and new anxiety, and to better manage hot flashes. It is also assumed that the user is on day 2 (in P1)) of her current cycle.

Scenario 1: User Wants to Move a Workout to an Earlier Day than Planned.

After completing the intake and first morning check in, the device application 222 provides the user with an overview of recommended workouts for the phase/week. On the current cycle day (day 2), user hormone levels, particularly estrogen, are low and as such, the device application 222 recommends a workout that is a low-intensity recovery session, including 25 minutes of yoga designed to open up the torso, enhance circulation, increase mental energy, and improve mobility. An alternative workout can include 15 minutes of easy animal flow movements, with the same goal. The workout outlined for day 8 (in P3) is a higher workload kettlebell circuit, which is included in the weekly overview to help the user plan ahead.

But since the user likes kettlebell circuit workouts, the user attempts to move it forward to the current day. In this event, the device application 222 can deliver a message such as: “Looks like you're chomping at the bit for some higher intensity work! Your estrogen levels today are much lower than they will be on day 8, and while you may feel ready to take on that workout right now, you'll be better off with what we've designed for you today. If you decide to go for the kettlebell circuit, you may experience more fatigue later, impaired recovery, and will be more prone to injury than you would be on day 8. For those reasons, be sure to leave yourself room to do the moves correctly and give yourself adequate time to recover.” Based on this message to the user, it is assumed that the user accepts the guidance and saves the more intense workout for Day 8.

Scenario 2: User Rates a Workout Poorly, FESS Recommendations Adjusted Accordingly.

Day 8 arrives, and the user follows the kettlebell circuit workout. On day 9, device application 222 asks the user to rate the workout, which she rates as 1 star (i.e., not recommended). Device application 222 can request and obtain clarifying information from the user (e.g., with choices like “I completed the workout, but felt terrible during and after”, “I was unable to complete the workout”, “I did the workout and felt okay, but didn't enjoy it”). Based on the rating and clarifying information, device application 222 flags the workout to not be recommended again in the original form and creates a new workout to fulfill its goals in a way that satisfies this user's needs and preferences. Meanwhile, the user completes a morning check-in, and device application 222 produces a recommended workout for that day based on up-to-date user logs and collected bodily function data. For example, device application 222 for this sample day can generate a recommended workout of upper body strength training at intensity level 8 followed by 10 minutes of sprint intervals.

Scenario 3: User has Lower than Expected HRV, FESS Recommendations Changed to Encourage Recovery.

In this scenario, the user completes the recommended workout and eats according to the FESS recommendations, but unexpected stressors appear afterward. For example, the user may have an unexpected emergency deadline at her job for which she must work until 11:30 PM on her laptop. Later, the user consumes a half of a bottle of wine and watches a movie to destress. Not only was the user's sleep shortened, but it was also impaired by late blue light exposure, the interruption in routine, and the metabolic impacts of the alcohol. As a result, when Day 10 rolls around, the user's HRV is drastically lower than her baseline. The morning user logs in device application 222 also indicate a lower energy level. In response to this updated information, device application 222 can deliver a message such as the following: “In this phase, you should be able to crush a plyometric workout! And maybe you'll be able to tomorrow, but your HRV indicates that your body hasn't adequately recovered from yesterday. Your energy rating reflects that, too. Today, let's prioritize activities that will make sure you recover and are ready to roll again tomorrow. If you choose to go ahead with a plyometric workout, your already-high cortisol will likely spike, making it difficult to recover, get restorative sleep, and leaving you prone to injury. Get a 2-mile walk in today, followed by some mobility work.”

In each of these scenarios and any other use of applications 222 and 242, the recommendations generated preferably rely on a large volume and variety of data that are not possible for the user to collect or use. By implementing the FESS engine and the kNN index ML-algorithm, the device application 222 and the server application 242 can generate, provide, and refine recommendations in a way that is tailored to each user's daily needs and according to that user's ever-changing symptoms, bodily function data, and menstrual cycle.

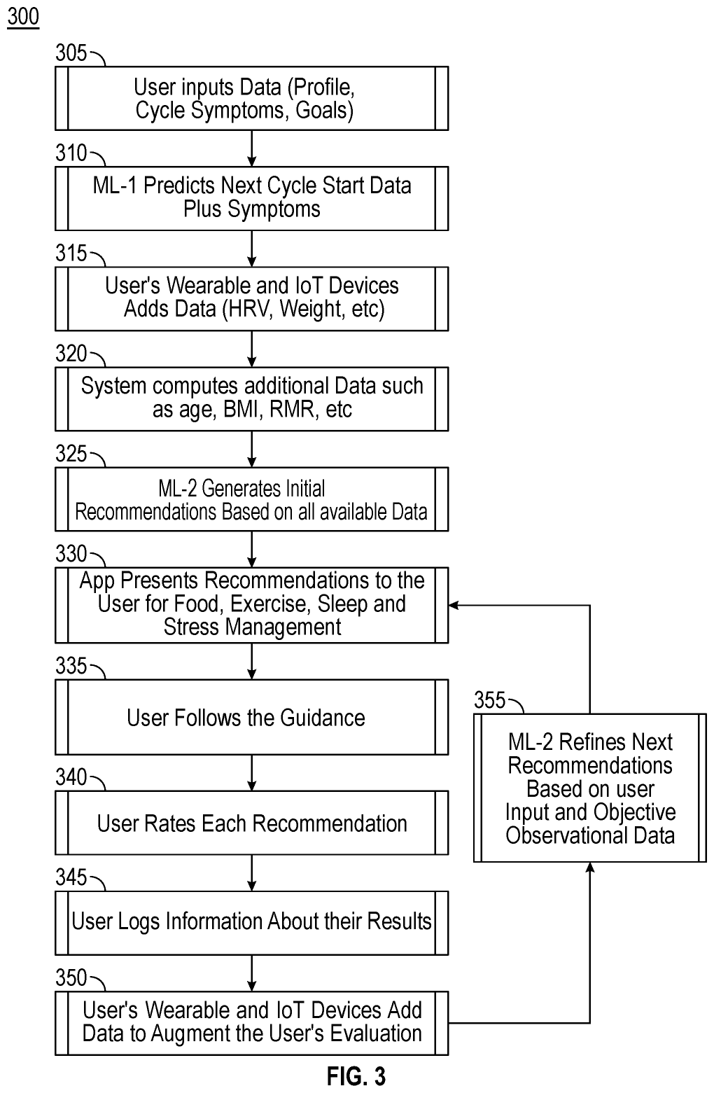

FIG. 3 is a flow chart for an analysis and recommendation process 300 according to an embodiment. As shown in FIG. 3, a user inputs data to the system (step 305). To initiate the input of data, the user can download device application 222 to the user's mobile device. Once downloaded, the user can provide user profile information, user-inputted data regarding food, exercise, sleep, and stress management, and recent menstrual cycle data (collectively, baseline user data). Further details on the types of baseline user data entered by the user are described above. In addition to this data, the baseline user data can also include bodily function data collected from the wearable device 200, the mobile device 220, and/or IoT devices.

Based on the baseline user data, a future menstrual cycle and/or the day or phase of a current menstrual cycle is determined (step 310). To make this determination, device application 222 on mobile device 220 can provide the baseline user data to server application 242 on server 240. Using the FESS engine and kNN index ML-algorithm, server application 242 uses the baseline user data including the past menstrual cycle data of the user and the large volume of data collected from other users to make accurate predictions of the length of the user's menstrual cycle and on what day and phase the user's cycle is currently situated.

In addition to collecting the user-inputted data, the bodily function data is collected (step 315). To enable this collection, the user can provide authorizations to enable device application 222 to collect and receive data from the user's wearable device 220, mobile device 200, and/or IoT devices. The data collected can be from the sensors in these devices and can include, for example, Heart Rate Variability (HRV), Resting Heart Rate (RHR), Exercise Minutes, Steps, Basal Body Temperature (BBT), Sleep [Random Eye Movement (REM) minutes, Non-Random Eye Movement (NREM) minutes], Sleep Quality, Weight, and Body Fat Percentage.

Additional data can be determined from the user-inputted data and/or collected bodily function data (step 320). For example, the device application 222 can take the user-inputted data and collected bodily function data and calculate values including HRV trends, User Cycle Day (based on standard cycle adjusted for this user's actual cycle length), Next Cycle Start Date, Sleep Debt, User Average Cycle Length, Menstrual Symptom Trends, Perimenopause Symptom Trends, and CS. Alternatively, mobile device 220 can send the data collected by device application 222 to server application 242 on server 240, and server application 242 can be configured to make these additional data determinations and calculations.

Initial recommendations for the user can then be generated (step 325). The initial set of recommendations can be based on the user baseline data including the user profile data, other user-inputted data (e.g., user preferences, symptoms, food, exercise, sleep, stress management), the bodily function data from wearable device 200, mobile device 220, and/or IoT devices, and the derived inputs based on the collected bodily function data (e.g., HRV, HRV trend, CS, CS trend, sleep debt, average cycle length, user's cycle data), the FESS engine, and the kNN index. The initial set of recommendations can be generated by server application 242 on server 240. Alternatively, the initial set of recommendations can be generated by device application 222 on mobile device 220.

The initial set of recommendations can be presented to the user (step 330). If the initial set of recommendations is generated by server application 242, server 240 can send the recommendations generated by server application 242 to mobile device 220, which provides the recommendations to device application 222 to present the recommendations to the user with requests for the user to follow the plan according to the recommendations. The recommendations can be presented to the user through a display on mobile device 220. Alternatively, the recommendations can be sent to the wearable device 200 and presented on a display of the wearable device 200.

After receiving the initial set of recommendations, the user follows the guidance (step 335). The guidance in the initial set of recommendations preferably includes food, exercise, sleep, and stress management recommendations. These recommendations can appear in a manner as shown above regarding an exemplary plan.

The user can enter ratings for each recommendation (step 340). The ratings can be entered through the device application 222 on mobile device 220. The ratings can be, for example, on a scale of 1-5 or 1-10 with 1 being low or very unsatisfactory and 5 or 10 being high or very satisfactory. In addition to entering ratings, the user can log information about the results (step 345). The results can include information contained in the exemplary logs described above comprising, for example, food intake, exercises performed, sleep related data, and stress management. The logged data can also include symptoms such as hot flashes, menstrual flow amount, and other symptom information. Device application 222 can be configured to ask for the ratings and logged data from the user at specific intervals of time, such as once in the morning after waking up and once in the evening before sleep. Alternatively, the user can elect at what time to enter the ratings and logged data.

Besides ratings and data input by the users, the bodily function data is also collected (step 350). As explained previously, the bodily function data can include HRV, RHR, exercise minutes, steps, BBT, sleep, sleep quality, weight, and body fat percentage, for example. This data can be detected and collected continuously and in real-time by device application 222.

The recommendations for the user can then be updated (step 355). Server application 242 (and/or device application 222) can be configured to provide new recommendations based on the user-entered ratings, logged data, and newly collected bodily function data. This additional data, as well as any additional data provided by other users contributing to the large volume of data available to the FESS engine and kNN index ML-algorithm, enable the server application 242 to provide updated recommendations that accurately reflect any changed circumstances, as well as provide improvements to the recommendations based on the machine learning. The process of providing the recommendations and receiving feedback (e.g., the user ratings, logged data, and newly collected bodily function data) can be continuously repeated over a certain period of time, such as daily, to enable the user to receive increasingly accurate and improved recommendations that account for the user's changing circumstances including food intake, exercise, sleep, stress management, and current day and phase of the user's menstrual cycle.

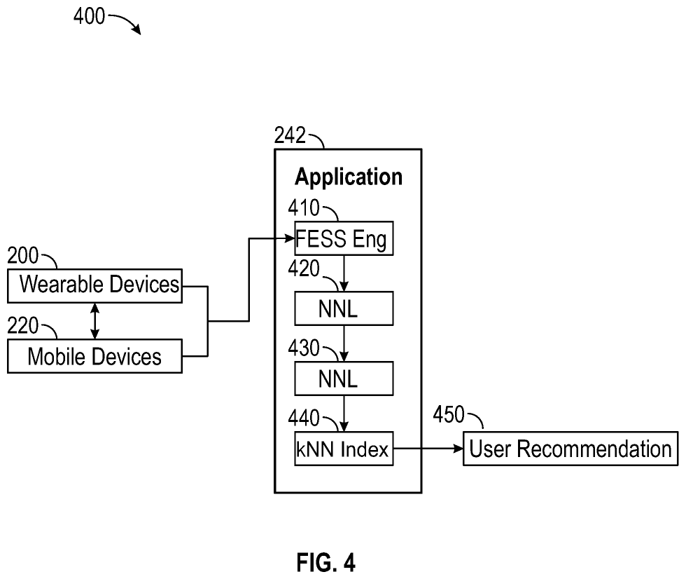

FIG. 4 is a functional block diagram of a recommendation application 400 for generating user recommendations 450 according to an embodiment. As shown in FIG. 4, the recommendation application 400 comprises wearable device 200, mobile device 220, and the server application 242. According to some embodiments, the server application 242 can include FESS engine 410, neural network layers (NNL) 420, 430, and a kNN index 440. As explained previously, FESS engine 410 can be a machine learning (ML)-based recommendation system that takes the user-inputted data along with other data sources including the bodily function data from wearable device 200, mobile device 220, and/or IoT devices and generates FESS recommendations tailored to the user for the areas of Food, Exercise, Sleep and Stress Management (FESS). The FESS engine 410 generates the FESS recommendations based on the user's history and trends for each of these areas (i.e., food, exercise, sleep, stress management), plus the user's hormone environment (e.g., based on the phase of the user's menstrual cycle), preferences and abilities. The FESS engine 410 also makes use of data regarding similar users in order to refine the FESS recommendations by using neural network layers (NNL) 420, 430 to make predictions for what will benefit the user the most during the time period covered by the user recommendation 450, for instance, a day or a week, as well as what the user will likely accept (i.e., likely that the user will perform the recommended plan) and rate highly. To facilitate the operation of the FESS engine 410, server application 242 can include a list of content items that the recommender system can assemble for food, exercise, sleep, and stress management recommendations.

The kNN index 440, according to an embodiment, is a machine learning algorithm that can be used to find clusters of similar items among a large volume of data collected from a large number of users based on commonalities and make predictions using that data. As more users use the system, server application 242 through FESS engine 410 and kNN index 440 can use similarity data to accelerate the arrival at the optimal set of recommendations for each user.

Using both the FESS engine 410 and the kNN index 440, server application 242 can generate an initial set of recommendations after the user sets up device application 222 and provides the requested initial information. The initial set of recommendations is based on the profile data, other user-inputted data (e.g., user preferences, symptoms, food, exercise, sleep, stress management), bodily function data from wearable device 200, mobile device 220, and/or IoT devices, derived inputs based on the collected bodily function data (e.g., HRV, HRV trend, CS, CS trend, sleep debt, average cycle length, user's cycle data), FESS engine 410, and kNN index 440. In addition, after receiving feedback from the user in response to applying the recommendations, server application 242 can generate updated recommendations based on a continuous loop that also takes into account the feedback and logs, FESS engine 410, kNN index 440, and the cycle norms vector.

Various embodiments of the invention are contemplated in addition to those disclosed hereinabove. The above-described embodiments should be considered as examples of the present invention, rather than as limiting the scope of the invention. In addition to the foregoing embodiments of the invention, review of the detailed description and accompanying drawings will show that there are other embodiments of the present invention. Accordingly, many combinations, permutations, variations, and modifications of the foregoing embodiments of the present invention not set forth explicitly herein will nevertheless fall within the scope of the present invention.

1. A method for analyzing detected bodily function information and user input information, comprising:
receiving first user input information input by the first user, the first user input information including at least a beginning date and an end date of a most recent menstrual cycle of the first user;
determining at least one of an upcoming menstrual cycle or a phase of an existing menstrual cycle of the first user based on the first user input information;
receiving first bodily function information from a wearable device worn by the first user, the wearable device configured to detect one or more bodily functions of the first user when worn by the first user;
generating at least one of a first food regimen or first exercise regimen for the first user based on the first bodily function information, the first user input information, and at least one of the determined upcoming menstrual cycle or the determined phase of the existing menstrual cycle;
receiving second user input information input by the user subsequent to receiving the first user input information;
receiving second bodily function information from the wearable device worn by the user subsequent to receiving the first bodily function information;
generating at least one of a second food regimen or second exercise regimen for the first user based on the second bodily function information, the second user input information, and at least one of the upcoming menstrual cycle or the phase of the existing menstrual cycle;
wherein the bodily function information includes data sufficient to determine at least a heart rate variability (HRV) of the first user.

2. A method according to claim 1, wherein the first bodily function information includes an HRV at a first level and the second bodily function information includes an HRV at a second level, different from the first level,
wherein the first exercise regimen is generated based on the HRV at the first level and the determined phase of the existing menstrual cycle, and
wherein the second exercise regimen for the first user is generated based on the HRV at the second level and the same determined phase of the existing menstrual cycle.

3. A method according to claim 2, wherein the second exercise regimen is different than the first exercise regimen based on the differences between the HRV at the first level and the HRV at the second level.

4. A method according to claim 3, wherein the second exercise regimen includes more exercise intensity commensurate with a higher capacity for activity than the first exercise regimen when the HRV at the first level is lower than the HRV at the second level, and
wherein the second exercise regimen includes less exercise intensity than the first exercise regimen when the HRV at the first level is higher than the HRV at the second level.

5. A method according to claim 1, wherein at least one of the upcoming menstrual cycle or the phase of the existing menstrual cycle is further determined based on historical bodily function information and historical user input information of a plurality of users other than the first user.

6. A method according to claim 5, further comprising:
providing the first user input information including the beginning date and the end date of the most recent menstrual cycle of the first user to at least one of a kNN index or ML-algorithm component;
providing the historical bodily function information and historical user input information of the plurality of users other than the first user to at least one of the kNN index or ML-algorithm component; and
identifying, using the kNN index or ML-algorithm component and the historical bodily function information and historical user input information of the plurality of users other than the first user, menstrual cycle data of the plurality of users other than the first user that are similar to the beginning date and the end date of the most recent menstrual cycle of the first user.

7. A method according to claim 6, wherein the at least one of the upcoming menstrual cycle or the phase of the existing menstrual cycle is further determined based on menstrual cycle data of the plurality of users other than the first user identified by the kNN index or ML algorithm component as being similar to the beginning date and the end date of the most recent menstrual cycle of the first user.

8. A method according to claim 1, wherein receiving the first bodily function information includes a basal body temperature (BBT) of the first user.

9. A method according to claim 1, wherein the wearable device worn by the first user is further configured to detect HRV, basal body temperature (BBT), and resting heartrate (RHR).

10. A method according to claim 9, wherein the first bodily function information includes at least HRV, BBT, and RHR detected by the wearable device worn by the first user.

11. A device, comprising:
a user interface for receiving user input information input by a first user;
a receiver for receiving bodily function information from a wearable device worn by the first user, the wearable device configured to detect one or more bodily functions of the first user when worn by the first user; and
a non-transitory computer readable storage device comprising a program that when executed by circuitry in the device configures the device to
determine at least one of an upcoming menstrual cycle or a phase of an existing menstrual cycle of the first user based on first user input information including at least a beginning date and an end date of a most recent menstrual cycle of the first user,
generate at least one of a first food regimen or a first exercise regimen for the first user based on first bodily function information received from the wearable device, the first user input information, and at least one of the determined upcoming menstrual cycle or the determined phase of the existing menstrual cycle, and
generate at least one of a second food regimen or second exercise regimen for the first user based on second bodily function information received from the wearable device subsequent to receiving the first bodily function information, second user input information input by the user subsequent to receiving the first user input information, and at least one of the upcoming menstrual cycle or the phase of the existing menstrual cycle,
wherein the bodily function information includes data sufficient to determine at least a heart rate variability (HRV) of the first user.

12. The device according to claim 11, wherein the program that when executed by circuitry in the device further configures the device to
generate the first exercise regimen based on the HRV of the first bodily function information being at a first level and the determined phase of the existing menstrual cycle, and
generate the second exercise regimen based on the HRV of the second bodily function information being at a second level, different from the first level, and the same determined phase of the existing menstrual cycle.

13. The device according to claim 12, wherein the second exercise regimen is different than the first exercise regimen based on the differences between the HRV at the first level and the HRV at the second level.

14. The device according to claim 13, wherein the second exercise regimen includes more exercise intensity commensurate with a higher capacity for activity than the first exercise regimen when the HRV at the first level is lower than the HRV at the second level, and
wherein the second exercise regimen includes less exercise intensity than the first exercise regimen when the HRV at the first level is higher than the HRV at the second level.

15. The device according to claim 12, wherein the determining at least one of the upcoming menstrual cycle or the phase of the existing menstrual cycle is further determined based on historical bodily function information and historical user input information of a plurality of users other than the first user.

16. A regimen recommendation system, comprising:
a wearable device comprising
a sensor configured to detect bodily functions of a first user when worn by the first user, and
a transmitter for transmitting bodily functions information of the first user to a network, wherein the bodily function information includes data sufficient to determine at least a heart rate variability (HRV) of the first user; and
a user device comprising
a user interface for receiving first user input information including at least a beginning date and an end date of a most recent menstrual cycle of the first user; and
a transmitter for transmitting the first user input information to the network; and
a network server, comprising:
a receiver configured to receive the user input information and first bodily function information of the first user from the network; and
a non-transitory computer readable storage device comprising a program that when executed by circuitry in the device configures the device to
determine at least one of an upcoming menstrual cycle or a phase of an existing menstrual cycle of the first user based on the user input information,
generate at least one of a first food regimen or a first exercise regimen for the first user based on the first bodily function information, the user input information, and at least one of the determined upcoming menstrual cycle or the determined phase of the existing menstrual cycle, and
generate at least one of a second food regimen or a second exercise regimen for the first user based on second bodily function information received from the network subsequent to receiving the first bodily function information, second user input information input by the user subsequent to receiving the first user input information, and at least one of the upcoming menstrual cycle or the phase of the existing menstrual cycle; and
a transmitter for transmitting a recommendation for the first user to the network, the recommendation including at least one of the first food regimen, the first exercise regimen, the second food regimen or the second exercise regimen.

17. The regimen recommendation system according to claim 16, wherein the first bodily function information includes an HRV at a first level and the second bodily function information includes an HRV at a second level, different from the first level,
wherein the first exercise regimen is generated based on the HRV at the first level and the determined phase of the existing menstrual cycle, and
wherein the second exercise regimen for the first user is generated based on the HRV at the second level and the same determined phase of the existing menstrual cycle.

18. The regimen recommendation system according to claim 16, wherein at least one of the upcoming menstrual cycle or the phase of the existing menstrual cycle is further determined based on historical bodily function information and historical user input information of a plurality of users other than the first user.

19. The regimen recommendation system according to claim 18, further comprising:
providing the first user input information to at least one of a kNN index or ML-algorithm component;
providing the historical bodily function information and historical user input information to at least one of the kNN index or ML-algorithm component; and
identifying, using the kNN index or ML-algorithm component and the historical bodily function information and historical user input information, menstrual cycle data of the plurality of users other than the first user that are similar to the beginning date and the end date of the most recent menstrual cycle of the first user.

20. The regimen recommendation system according to claim 19, wherein the at least one of the upcoming menstrual cycle or the phase of the existing menstrual cycle is further determined based on menstrual cycle data identified by the kNN index or ML algorithm component.
